(19) United States
(12) Patent Application Publication
(10) Pub. No.: US 2014/0316207 A1
(43) Pub. Date: Oct. 23, 2014

(54) Platform for Laryngeal Microsurgery

(71) Applicant: MOR RESEARCH APPLICATIONS LTD., Tel Aviv (IL)

(72) Inventor: Moshe Hain, Jerusalem (IL)

(21) Appl. No.: 14/114,692

(22) PCT Filed: May 8, 2012
(86) PCT No.: PCT/IL2012/000184

§ 371 Date: Dec. 19, 2013

Related U.S. Application Data
(60) Provisional application No. 61/483,760, filed on May 9, 2011.

(51) Int. Cl. A61B 1/267; A61B 1/00

(52) U.S. Cl. CPC A61B 1/267; A61B 1/00073; A61B 1/00082; A61B 1/00105
USPC 600/194

(57) ABSTRACT

A flexible laryngoscope (10) is disclosed, comprising a flexible body with a plurality of longitudinal channels (150) therethrough, an external groove (110) for an intubation tube, and two inflatable zones (120,130). The laryngoscope is adapted to be inserted intra-orally and to sit in place in the upper hypopharynx and oral cavity in a bent fashion. The laryngoscope is used as a platform for microsurgery by passing flexible microsurgery tools through the channels. In preferred embodiments of the invention, the laryngoscope body comprises a flexible corrugated intermediate portion (102) that allows the body to be bent to fit within the patient's hypopharynx and oral cavity. The inflatable zones are adapted to allow the laryngoscope to be fixed in place when they are inflated.

### FIELD OF THE INVENTION

This invention relates to devices adapted for use in laryngeal microsurgery. In particular, it relates to a flexible modular platform for use in such surgery.

### BACKGROUND OF THE INVENTION

The surgical method most commonly used today for procedures on the larynx and hypopharynx is Direct Laryngoscopy. This method enables both direct view of the region upon which the procedure is being performed via the use of a binocular microscope and the use of a variety of surgical instruments.

Due to the anatomy of the pharynx and its different parts, as well as due to the structure of the human neck, the position in which the patient must be placed during surgery is unnatural. In order to obtain the correct angle and to enable a direct view of the throat, it is necessary to place significant external pressure on the tongue, teeth, and the cartilage of the throat, and thus insertion of the heavy surgical instrument can damage delicate tissues such as the mucous membranes.

In many cases, it is necessary to struggle during a period of minutes in order to expose particular regions of interest. For example, the epiglottis and the base of the tongue are particularly difficult to expose. Some areas of the larynx such as the inferior aspect of the vocal fold and the vestibule are hidden from view. It is extremely difficult to obtain a view of these areas and to operate upon them using direct laryngoscopy. The difficulty of obtaining a view in these instances can lead to the problem of damage to delicate tissues during the handling of the surgical instruments.

A number of inventions disclosing means for positioning surgical devices for use in laryngeal surgery are known. U.S. Pat. No. 5,894,840 and European Pat. No. EP1062963 are examples of disclosures of means for fixing an endotracheal tube. These devices ensure that the endotracheal tube will remain in place and the patient's airway will remain open during surgery. U.S. Pat. No. 4,289,128 discloses a laryngeal tube for use in microsurgery. It provides a tube with an inflatable cuff and a ventilation channel. U.S. Pat. No. 7,273,053 discloses a device for monitoring and controlling a laryngeal mask airway device. Air pressure in the reversibly inflatable seal ring or cuff of a laryngeal mask airway device is corrected as necessary to conform to a predetermined pressure level.

A laryngoscope that will allow passage of microsurgical tools and maintain the patient's airway but that allows the patient to remain in a natural body position and that helps prevent the risk of tissue damage common in typical laryngoscopic procedures thus remains a long-felt, yet unmet, need.

### SUMMARY OF THE INVENTION

The present invention is designed to meet this long-felt need. A flexible laryngoscope is disclosed that is able to bend according to the anatomy of a particular patient. The laryngoscope is designed for trans-oral insertion and enables performance of surgery on the larynx while the patient remains in a natural position and without the need for application of external force that may damage delicate tissues.

It is therefore an object of the present invention to provide a flexible laryngoscope, wherein said flexible laryngoscope comprises:
a flexible body comprising a plurality of longitudinal channels extending therethrough and at least one longitudinal groove in outer circumference of said body, said longitudinal groove adapted to accommodate an intubation tube;
a proximal inflatable zone adapted for inflation and deflation; said proximal inflatable zone disposed about the outer circumference of said body near the proximal end of said body, wherein the outer diameter of said proximal inflatable zone is no larger than that of said body when said proximal inflatable zone is in its deflated state, and further wherein said proximal inflatable zone is adapted to inflate to a predetermined outer circumference at least large enough to contact the inner walls of the hypopharynx of a patient;
a distal inflatable zone adapted for inflation and deflation, said distal inflatable zone disposed about the outer circumference of said body near the distal end of said body, wherein the outer diameter of said distal inflatable zone is no larger than that of said body when said distal inflatable zone is in its deflated state, and further wherein said distal inflatable zone is adapted to inflate to a predetermined outer circumference at least large enough to contact the inner walls of the hypopharynx of a patient.

It is another object of the present invention to provide the flexible laryngoscope as defined above, wherein said distal inflatable zone comprises at least one balloon.

It is another object of the present invention to provide the flexible laryngoscope as defined above, wherein said proximal inflatable zone comprises at least one balloon.

It is another object of the present invention to provide the flexible laryngoscope as defined above, wherein said distal inflatable zone comprises at least two separate balloons, each of which is adapted to inflate so as to encircle part of said circumference.

It is another object of the present invention to provide the flexible laryngoscope as defined above, wherein said proximal inflatable zone comprises at least two separate balloons, each of which is adapted to inflate so as to encircle part of said circumference.

It is another object of the present invention to provide the flexible laryngoscope as defined above, wherein said proximal and distal inflatable zones are integral with said body of said laryngoscope.

It is another object of the present invention to provide the flexible laryngoscope as defined above, wherein substantially all of said body of said flexible laryngoscope comprises at least one inflatable zone.

It is another object of the present invention to provide the flexible laryngoscope as defined above, wherein substantially all of said flexible laryngoscope comprises at least one inflatable zone.

It is another object of the present invention to provide the flexible laryngoscope as defined above, additionally comprising a proximal inflation inlet located at the proximal end of said body, said proximal inflation inlet connectable to an external source of fluid.

It is another object of the present invention to provide the flexible laryngoscope as defined above, additionally comprising a distal inflation inlet located at the proximal end of said body, said distal inflation inlet connectable to an external source of fluid.

It is another object of the present invention to provide the flexible laryngoscope as defined above, wherein said external source of fluid is pressurized.

It is another object of the present invention to provide the flexible laryngoscope as defined above, wherein said fluid is selected from a group consisting of air, nitrogen, argon, water, saline solution, anesthetic, analgesic, antibiotic, preservative, anti-caking agent, anti-sticking agent, and any combination thereof.

It is another object of the present invention to provide the flexible laryngoscope as defined above, additionally comprising at least one first fluid connection channel providing a fluid connection between said proximal inflatable zone and said proximal inflation inlet.

It is another object of the present invention to provide the flexible laryngoscope as defined above, additionally comprising at least one second fluid connection channel providing a fluid connection between said distal inflatable zone and said distal inflation inlet.

It is another object of the present invention to provide the flexible laryngoscope as defined above, wherein cross-sectional shape of said body comprises at least one selected from a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the flexible laryngoscope as defined above, wherein n is less than about 20.

It is another object of the present invention to provide the flexible laryngoscope as defined above, wherein said cross-sectional shape differs in different portions of said body.

It is another object of the present invention to provide the flexible laryngoscope as defined above, wherein diameter of said body differs in different portions of said body.

It is another object of the present invention to provide the flexible laryngoscope as defined above, wherein said diameter is greatest at said proximal end and smallest at said distal end of said body.

It is another object of the present invention to provide the flexible laryngoscope as defined above, wherein said diameter is greater in at least one of said proximal end and said distal end of said body than in at least some portion of central region of said body.

It is another object of the present invention to provide the flexible laryngoscope as defined above, wherein said longitudinal channels have cross-section selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the flexible laryngoscope as defined above, wherein cross-section of said longitudinal channels differs in shape in different portions of said body.

It is another object of the present invention to provide the flexible laryngoscope as defined above, wherein cross-section of said longitudinal channels differs in size in different portions of said body.

It is another object of the present invention to provide the flexible laryngoscope as defined above, wherein said laryngoscope is modular.

It is another object of the present invention to provide the flexible laryngoscope as defined above, wherein said modules are selected from a group consisting of proximal portion modules, distal portion modules and body portion modules.

It is another object of the present invention to provide the flexible laryngoscope as defined above, wherein said modules differ in size.

It is another object of the present invention to provide the flexible laryngoscope as defined above, wherein said modules differ in shape.

It is another object of the present invention to provide the flexible laryngoscope as defined above, adapted to be inserted intra-orally and to be placed, while in use, partially within the upper hypopharynx and partially within the oral cavity, wherein said flexible laryngoscope is further adapted to sit in place in a bent fashion.

It is another object of the present invention to provide the flexible laryngoscope as defined above, wherein said flexible body comprises two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions.

It is another object of the present invention to provide the flexible laryngoscope as defined above, wherein said intermediate portion is formed in an accordion shape.

It is another object of the present invention to provide the flexible laryngoscope as defined above, wherein said plurality of longitudinal channels comprises a central channel and at least four side channels.

It is another object of the present invention to provide the flexible laryngoscope as defined above, wherein said central channel is adapted to accommodate an endoscopic camera and/or light source.

It is another object of the present invention to provide the flexible laryngoscope as defined above, wherein at least one of said channels is adapted to accommodate at least one optical fiber so as to transmit light to the distal part of said flexible laryngoscope.

It is another object of the present invention to provide the flexible laryngoscope as defined above, wherein at least one said plurality of longitudinal channels further comprises an O-ring groove about its inner circumference substantially near the proximal end.

It is another object of the present invention to provide the flexible laryngoscope as defined above, wherein each of said plurality of longitudinal channels further comprises a locking mechanism with gradable friction levels.

It is another object of the present invention to provide the flexible laryngoscope as defined above, wherein at least one of said plurality of longitudinal channels is adapted to accommodate at least one surgical tool selected from a group consisting of scissors, forceps, injection needle, dissector, laser, suction, fiber optics, and any other required surgical instrument.

It is another object of the present invention to provide the flexible laryngoscope as defined above, additionally comprising a locking mechanism adapted to fixate the orientation and angle of said flexible body.

It is another object of the present invention to provide the flexible laryngoscope as defined above, wherein said locking mechanism is an integral part of said laryngoscope.

It is another object of the present invention to provide the flexible laryngoscope as defined above, wherein said locking mechanism is coupled to said laryngoscope by means selected from a group consisting of mechanical means glue, vacuum, clips, magnetic means, electrical means and any combination thereof.

It is another object of the present invention to provide the flexible laryngoscope as defined above, wherein at least one of said distal, proximal and body inflatable zones is enabled to be translated along the longitudinal axis of said laryngoscope.

It is another object of the present invention to provide the flexible laryngoscope as defined above, wherein said at least one translatable inflatable zone rides in grooves in said body of said laryngoscope.

It is another object of the present invention to provide the flexible laryngoscope as defined above, wherein said grooves are disposed on said body of said laryngoscope in at least one manner selected from a group consisting of longitudinally and spirally.

It is another object of the present invention to provide a laryngoscope system, comprising:
a flexible laryngoscope as defined above; and,
a stand adapted to accommodate simultaneously the proximal ends of a plurality of surgical tools, said tools enabled to be passed through the channels of the flexible laryngoscope.

It is another object of the present invention to provide the laryngoscope system as defined above, wherein said stand is free-standing.

It is another object of the present invention to provide the laryngoscope system as defined above, wherein said stand is adapted to be anchored to a surgical bed, and further comprising anchoring means for anchoring said stand to said surgical bed.

It is another object of the present invention to provide the laryngoscope system as defined above, further comprising a screen adapted to display an optical signal from an endoscopic camera being used in conjunction with said laryngoscope.

It is another object of the present invention to provide a method for using a flexible laryngoscope, comprising:
obtaining a flexible laryngoscope comprising:
   a flexible body comprising a plurality of longitudinal channels extending therethrough and at least one longitudinal groove in outer circumference of said body, said longitudinal groove adapted to accommodate an intubation tube;
   a proximal inflatable zone adapted for inflation and deflation, said proximal inflatable zone disposed about the outer circumference of said body near the proximal end of said body, wherein the outer diameter of said proximal inflatable zone is no larger than that of said body when said proximal inflatable zone is in its deflated state, and further wherein said proximal inflatable zone is adapted to inflate to a predetermined outer circumference at least large enough to contact the inner walls of the hypopharynx of a patient;
   a distal inflatable zone adapted for inflation and deflation, said distal inflatable zone disposed about the outer circumference of said body near the distal end of said body, wherein the outer diameter of said distal inflatable zone is no larger than that of said body when said distal inflatable zone is in its deflated state, and further wherein said distal inflatable zone is adapted to inflate to a predetermined outer circumference at least large enough to contact the inner walls of the hypopharynx of a patient;
   a proximal inflation inlet located at the proximal end of said body, said proximal inflation inlet connectable to an external source of fluid;
   a distal inflation inlet located at the proximal end of said body, said distal inflation inlet connectable to an external source of fluid;
   a first fluid connection channel providing a fluid connection between said proximal inflatable zone and said proximal inflation inlet; and,
   a second fluid connection channel providing a fluid connection between said distal inflatable zone and said distal inflation inlet;
inserting said laryngoscope trans-orally into the oral cavity and hypopharynx of a patient;
intubating said patient by inserting a endotracheal breathing tube and then gliding the laryngoscope into place with the endotracheal tube located in said longitudinal groove;
connecting each of said inflation zones via said fluid connection channels and inflation inlets to at least one source of fluid;
inflating said inflation zones with said at least one fluid; and,
inserting at least one device selected from a group consisting of (a) an endoscopic camera, (b) a light source, and (c) at least one micro surgical instrument through at least one of said longitudinal channels.

It is another object of the present invention to provide the method as defined above, wherein said distal inflatable zone comprises at least one balloon.

It is another object of the present invention to provide the method as defined above, wherein said proximal inflatable zone comprises at least one balloon.

It is another object of the present invention to provide the method as defined above, wherein said distal inflatable zone comprises at least two separate balloons, each of which is adapted to inflate so as to encircle part of said circumference.

It is another object of the present invention to provide the method as defined above, wherein said proximal inflatable zone comprises at least two separate balloons, each of which is adapted to inflate so as to encircle part of said circumference.

It is another object of the present invention to provide the method as defined above, wherein said proximal and distal inflatable zones are integral with said body of said laryngoscope.

It is another object of the present invention to provide the method as defined above, wherein substantially all of said body of said flexible laryngoscope comprises at least one inflatable zone.

It is another object of the present invention to provide the method as defined above, wherein substantially all of said flexible laryngoscope comprises at least one inflatable zone.

It is another object of the present invention to provide the method as defined above, additionally comprising a locking mechanism adapted to fixate at least one selected from a group consisting of (a) at least one of said channels; (b) said proximal inflatable zone; (c) said distal inflatable zone; (d) said entire laryngoscope; and any combination thereof.

It is another object of the present invention to provide the method as defined above, wherein each of said plurality of longitudinal channels further comprises a locking mechanism with gradable friction levels, said method further comprising a step of locking at least one of said locking mechanisms, thereby fixing the position of the device inserted through said longitudinal channel.

It is another object of the present invention to provide the method as defined above, additionally comprising step of providing said cross-sectional shape of said body selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the method as defined above, wherein n is less than about 20.

It is another object of the present invention to provide the method as defined above, additionally comprising step of providing a different said cross-sectional shape in different portions of said body.

It is another object of the present invention to provide the method as defined above, additionally comprising step of providing a different diameter of said body in different portions of said body.

It is another object of the present invention to provide the method as defined above, additionally comprising step of providing said body with said diameter greatest at said proximal end of said body and smallest at said distal end of said body.

It is another object of the present invention to provide the method as defined above, additionally comprising step of providing said body with said diameter greater in at least one of said proximal end and said distal end of said body than in at least some portion of central region of said body.

It is another object of the present invention to provide the method as defined above, additionally comprising step of selecting said fluid from a group consisting of air, nitrogen, argon, water, saline solution, anesthetic, analgesic, antibiotic, preservative, anti-caking agent, anti-sticking agent, and any combination thereof.

It is another object of the present invention to provide the method as defined above, additionally comprising step of providing said inflation fluid as a pressurized gas.

It is another object of the present invention to provide the method as defined above, additionally comprising step of providing said longitudinal channels with cross-section selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the method as defined above, additionally comprising step of providing cross-section of said longitudinal channels differing in shape in different portions of said body.

It is another object of the present invention to provide the method as defined above, additionally comprising step of providing cross-section of said longitudinal channels differing in size in different portions of said body.

It is another object of the present invention to provide the method as defined above, additionally comprising a step of assembling said flexible laryngoscope from modules.

It is another object of the present invention to provide the method as defined above, wherein said modules are selected from a group consisting of proximal portion modules, distal portion modules and body portion modules.

It is another object of the present invention to provide the method as defined above, wherein said modules differ in size.

It is another object of the present invention to provide the method as defined above, wherein said modules differ in shape.

It is another object of the present invention to provide the method as defined above, further comprising a step of fixing said laryngoscope to a stand adapted to accommodate simultaneously the proximal ends of a plurality of surgical tools, said tools enabled to be passed through the channels of the flexible laryngoscope.

It is another object of the present invention to provide the method as defined above, further comprising a step of displaying on a screen an optical signal from an endoscopic camera being used in conjunction with said laryngoscope.

It is another object of the present invention to provide the method as defined above, additionally comprising step of inserting said laryngoscope trans-orally and placing, while in use, partially within the upper hypopharynx and partially within the oral cavity, wherein said flexible laryngoscope is further adapted to sit in place in a bent fashion.

It is another object of the present invention to provide the method as defined above, additionally comprising step of providing said flexible body with two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions.

It is another object of the present invention to provide the method as defined above, additionally comprising step of providing said intermediate portion in an accordion shape.

It is another object of the present invention to provide the method as defined above, additionally comprising step of providing said plurality of longitudinal channels with a central channel and four side channels.

It is another object of the present invention to provide the method as defined above, additionally comprising step of providing said central channel so as to accommodate at least one of an endoscopic camera and a light source.

It is another object of the present invention to provide the method as defined above, wherein at least one of said channels is adapted to accommodate at least one optical fiber so as to transmit light to the distal part of said flexible laryngoscope.

It is another object of the present invention to provide the method as defined above, additionally comprising step of providing at least one said plurality of longitudinal channels with an O-ring groove about its inner circumference substantially near the proximal end.

It is another object of the present invention to provide the method as defined above, additionally comprising step of providing each of said plurality of longitudinal channels with a locking mechanism with gradable friction levels.

It is another object of the present invention to provide the method as defined above, additionally comprising step of providing at least one of said plurality of longitudinal channels so as to accommodate at least one surgical tool selected from a group consisting of scissors, forceps, injection needle, dissector, laser, suction, fiber optics and any other required surgical instrument.

It is another object of the present invention to provide the method as defined above, additionally comprising step of providing said laryngoscope with a locking mechanism adapted to fixate the orientation and angle of said flexible body.

It is another object of the present invention to provide the method as defined above, additionally comprising step of providing said locking mechanism is an integral part of said laryngoscope.

It is another object of the present invention to provide the method as defined above, additionally comprising step of coupling said locking mechanism to said laryngoscope by means selected from a group consisting of mechanical means glue, vacuum, clips, magnetic means, electrical means and any combination thereof.

It is another object of the present invention to provide the method as defined above, additionally comprising a step of translating at least one of said distal, proximal and body inflatable zones along the longitudinal axis of said laryngoscope.

It is another object of the present invention to provide the method as defined above, wherein said at least one translatable inflatable zone rides in grooves in said body of said laryngoscope.

It is another object of the present invention to provide the method as defined above, additionally comprising a step of disposing said grooves on said body of said laryngoscope in at least one manner selected from a group consisting of longitudinally and spirally.

It is another object of the present invention to provide a flexible port for use during a laparoscopic surgery, wherein said flexible port comprises:
a flexible body comprising a plurality of longitudinal channels extending therethrough;
at least two inflatable zones adapted for inflation and deflation; one said inflatable zone distal to the other said inflatable zone, said inflatable zones disposed about the outer circumference of said body wherein the outer diameter of said distal inflatable zone is no larger than that of said body when said distal inflatable zone is in its deflated state, and further wherein said proximal and distal inflatable zones are adapted to inflate to a predetermined outer circumference at least large enough to retain said flexible port in a substantially fixed position relative to a patient's body.

It is another object of the present invention to provide the flexible laryngoscope as defined above, wherein said distal inflatable zone comprises at least one balloon.

It is another object of the present invention to provide the flexible laryngoscope as defined above, wherein said proximal inflatable zone comprises at least one balloon.

It is another object of the present invention to provide the flexible laryngoscope as defined above, wherein said distal inflatable zone comprises at least two separate balloons, each of which is adapted to inflate so as to encircle part of said circumference.

It is another object of the present invention to provide the flexible laryngoscope as defined above, wherein said proximal inflatable zone comprises at least two separate balloons, each of which is adapted to inflate so as to encircle part of said circumference.

It is another object of the present invention to provide the flexible laryngoscope as defined above, wherein said proximal and distal inflatable zones are integral with said body of said port.

It is another object of the present invention to provide the flexible laryngoscope as defined above, wherein substantially all of said body of said flexible port comprises at least one inflatable zone.

It is another object of the present invention to provide the flexible laryngoscope as defined above, wherein substantially all of said flexible port comprises at least one inflatable zone.

It is another object of the present invention to provide the flexible laryngoscope as defined above, additionally comprising a proximal inflation inlet located at the proximal end of said body, said proximal inflation inlet connectable to an external source of pressurized fluid.

It is another object of the present invention to provide the flexible laryngoscope as defined above, additionally comprising a distal inflation inlet located at the proximal end of said body, said distal inflation inlet connectable to an external source of fluid.

It is another object of the present invention to provide the flexible laryngoscope as defined above, wherein said external source of fluid is pressurized.

It is another object of the present invention to provide the flexible laryngoscope as defined above, wherein said fluid is selected from a group consisting of air, nitrogen, argon, water, saline solution, anesthetic, analgesic, antibiotic, preservative, anti-caking agent, anti-sticking agent, and any combination thereof.

It is another object of the present invention to provide the flexible laryngoscope as defined above, additionally comprising at least one first fluid connection channel providing a fluid connection between said proximal inflatable zone and said proximal inflation inlet.

It is another object of the present invention to provide the flexible laryngoscope as defined above, additionally comprising at least one second fluid connection channel providing a fluid connection between said distal inflatable zone and said distal inflation inlet.

It is another object of the present invention to provide the flexible laryngoscope as defined above, wherein cross-sectional shape of said body comprises at least one selected from a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the flexible laryngoscope as defined above, wherein n is less than about 20.

It is another object of the present invention to provide the flexible laryngoscope as defined above, wherein said cross-sectional shape differs in different portions of said body.

It is another object of the present invention to provide the flexible laryngoscope as defined above, wherein diameter of said body differs in different portions of said body.

It is another object of the present invention to provide the flexible laryngoscope as defined above, wherein said diameter is greatest at said proximal end and smallest at said distal end of said body.

It is another object of the present invention to provide the flexible laryngoscope as defined above, wherein said diameter is greater in at least one of said proximal end and said distal end of said body than in at least some portion of central region of said body.

It is another object of the present invention to provide the flexible laryngoscope as defined above, wherein said longitudinal channels have cross-section selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the flexible laryngoscope as defined above, wherein cross-section of said longitudinal channels differs in shape in different portions of said body.

It is another object of the present invention to provide the flexible laryngoscope as defined above, wherein cross-section of said longitudinal channels differs in size in different portions of said body.

It is another object of the present invention to provide the flexible laryngoscope as defined above, wherein said port is modular.

It is another object of the present invention to provide the flexible laryngoscope as defined above, wherein said modules are selected from a group consisting of proximal portion modules, distal portion modules and body portion modules.

It is another object of the present invention to provide the flexible laryngoscope as defined above, wherein said modules differ in size.

It is another object of the present invention to provide the flexible laryngoscope as defined above, wherein said modules differ in shape.

It is another object of the present invention to provide the flexible laryngoscope as defined above, adapted to be inserted intra-orally and to be placed, while in use, partially within the upper hypopharynx and partially within the oral cavity, wherein said flexible port is further adapted to sit in place in a bent fashion.

It is another object of the present invention to provide the flexible laryngoscope as defined above, wherein said flexible body comprises two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions.

It is another object of the present invention to provide the flexible laryngoscope as defined above, wherein said intermediate portion is formed in an accordion shape.

It is another object of the present invention to provide the flexible laryngoscope as defined above, wherein said plurality of longitudinal channels comprises a central channel and four side channels.

It is another object of the present invention to provide the flexible laryngoscope as defined above, wherein said central channel is adapted to accommodate at least one of an endoscopic camera and a light source.

It is another object of the present invention to provide the flexible laryngoscope as defined above, wherein at least one of said channels is adapted to accommodate at least one optical fiber so as to transmit light to the distal part of said flexible port.

It is another object of the present invention to provide the flexible laryngoscope as defined above, wherein at least one said plurality of longitudinal channels further comprises an O-ring groove about its inner circumference substantially near the proximal end.

It is another object of the present invention to provide the flexible laryngoscope as defined above, wherein each of said plurality of longitudinal channels further comprises a locking mechanism with gradable friction levels.

It is another object of the present invention to provide the flexible laryngoscope as defined above, wherein at least one of said plurality of longitudinal channels is adapted to accommodate at least one surgical tool selected from a group consisting of scissors, forceps, injection needle, dissector, laser, suction, fiber optics, and any other required surgical instrument.

It is another object of the present invention to provide the flexible laryngoscope as defined above, additionally comprising a locking mechanism adapted to fixate the orientation and angle of said flexible body.

It is another object of the present invention to provide the flexible laryngoscope as defined above, wherein said locking mechanism is an integral part of said port.

It is another object of the present invention to provide the flexible laryngoscope as defined above, wherein said locking mechanism is coupled to said port by means selected from a group consisting of mechanical means glue, vacuum, clips, magnetic means, electrical means and any combination thereof.

It is another object of the present invention to provide the flexible laryngoscope as defined above, additionally comprising a locking mechanism adapted to fixate at least one selected from a group consisting of (a) at least one of said channels; (b) said proximal inflatable zone; (c) said distal inflatable zone; (d) said entire port; and any combination thereof.

It is another object of the present invention to provide the flexible laryngoscope as defined above, wherein at least one of said distal, proximal and body inflatable zones is enabled to be translated along the longitudinal axis of said laryngoscope.

It is another object of the present invention to provide the flexible laryngoscope as defined above, wherein said at least one translatable inflatable zone rides in grooves in said body of said laryngoscope.

It is another object of the present invention to provide the flexible laryngoscope as defined above, wherein said grooves are disposed on said body of said laryngoscope in at least one manner selected from a group consisting of longitudinally and spirally.

It is another object of the present invention to provide a port system, comprising:
a flexible port as defined above; and,
a stand adapted to accommodate simultaneously the proximal ends of a plurality of surgical tools to be passed through the channels of the flexible laryngoscope, said tools enabled to be passed through the channels of the flexible port.

It is another object of the present invention to provide the port system as defined above, wherein said stand is free-standing.

It is another object of the present invention to provide the port system as defined above, wherein said stand is adapted to be anchored to a surgical bed, and further comprising anchoring means for anchoring said stand to said surgical bed.

It is another object of the present invention to provide the port system as defined above, further comprising a screen adapted to display an optical signal from an endoscopic camera being used in conjunction with said port.

It is another object of the present invention to provide a method for using a flexible port, comprising:
obtaining a flexible port comprising:
   a flexible body comprising a plurality of longitudinal channels extending therethrough;
   at least two inflatable zones adapted for inflation and deflation; one said inflatable zone distal to the other said inflatable zone, said inflatable zones disposed about the outer circumference of said body wherein the outer diameter of said distal inflatable zone is no larger than that of said body when said distal inflatable zone is in its deflated state, and further wherein said proximal and distal inflatable zones are adapted to inflate to a predetermined outer circumference at least large enough to retain said flexible port in a substantially fixed position relative to a patient's body;
   a proximal inflation inlet located at the proximal end of said body, said proximal inflation inlet connectable to an external source of fluid;
   a distal inflation inlet located at the proximal end of said body, said distal inflation inlet connectable to an external source of fluid;
   a first fluid connection channel providing a fluid connection between said proximal inflatable zone and said proximal inflation inlet; and,
   a second fluid connection channel providing a fluid connection between said distal inflatable zone and said distal inflation inlet;
inserting said port into a body cavity [symbol] of patient;
connecting each of said inflation zones via said fluid connection channels and inflation inlets to at least one source of fluid;
inflating said inflation zones with said at least one fluid; and,
inserting at least one device selected from a group consisting of (a) an endoscopic camera, (b) a light source, and (c) at least one microsurgical instrument through at least one of said longitudinal channels.

It is another object of the present invention to provide the method as defined above, wherein said distal inflatable zone comprises at least one balloon.

It is another object of the present invention to provide the method as defined above, wherein said proximal inflatable zone comprises at least one balloon.

It is another object of the present invention to provide the method as defined above, wherein said distal inflatable zone comprises at least two separate balloons, each of which is adapted to inflate so as to encircle part of said circumference.

It is another object of the present invention to provide the method as defined above, wherein said proximal inflatable zone comprises at least two separate balloons, each of which is adapted to inflate so as to encircle part of said circumference.

It is another object of the present invention to provide the method as defined above, wherein said proximal and distal inflatable zones are integral with said body of said port.

It is another object of the present invention to provide the method as defined above, wherein substantially all of said body of said flexible port comprises at least one inflatable zone.

It is another object of the present invention to provide the method as defined above, wherein substantially all of said flexible port comprises at least one inflatable zone.

It is another object of the present invention to provide the method as defined above, additionally comprising a locking mechanism adapted to fixate at least one selected from a group consisting of (a) at least one of said channels; (b) said proximal inflatable zone; (c) said distal inflatable zone; (d) said entire port; and any combination thereof.

It is another object of the present invention to provide the method as defined above, wherein each of said plurality of longitudinal channels further comprises a locking mechanism with gradable friction levels, said method further comprising a step of locking at least one of said locking mechanisms, thereby fixing the position of the device inserted through said longitudinal channel.

It is another object of the present invention to provide the method as defined above, additionally comprising step of providing said cross-sectional shape of said body selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the method as defined above, wherein n is less than about 20.

It is another object of the present invention to provide the method as defined above, additionally comprising step of providing a different said cross-sectional shape in different portions of said body.

It is another object of the present invention to provide the method as defined above, additionally comprising step of providing a different diameter of said body in different portions of said body.

It is another object of the present invention to provide the method as defined above, additionally comprising step of providing said body with said diameter greatest at said proximal end of said body and smallest at said distal end of said body.

It is another object of the present invention to provide the method as defined above, additionally comprising step of providing said body with said diameter greater in at least one of said proximal end and said distal end of said body than in at least some portion of central region of said body.

It is another object of the present invention to provide the method as defined above, additionally comprising step of selecting said fluid from a group consisting of air, nitrogen, argon, water, saline solution, anesthetic, analgesic, antibiotic, preservative, anti-caking agent, anti-sticking agent, and any combination thereof.

It is another object of the present invention to provide the method as defined above, additionally comprising step of providing said inflation fluid as a pressurized gas.

It is another object of the present invention to provide the method as defined above, additionally comprising step of providing said longitudinal channels with cross-section selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the method as defined above, additionally comprising step of providing cross-section of said longitudinal channels differing in shape in different portions of said body.

It is another object of the present invention to provide the method as defined above, additionally comprising step of providing cross-section of said longitudinal channels differing in size in different portions of said body.

It is another object of the present invention to provide the method as defined above, additionally comprising a step of assembling said flexible port from modules.

It is another object of the present invention to provide the method as defined above, wherein said modules are selected from a group consisting of proximal portion modules, distal portion modules and body portion modules.

It is another object of the present invention to provide the method as defined above, wherein said modules differ in size.

It is another object of the present invention to provide the method as defined above, wherein said modules differ in shape.

It is another object of the present invention to provide the method as defined above, further comprising a step of fixing said port to a stand adapted to accommodate simultaneously the proximal ends of a plurality of surgical tools to be passed through the channels of the flexible laryngoscope, said tools enabled to be passed through the channels of the flexible port.

It is another object of the present invention to provide the method as defined above, further comprising a step of displaying on a screen an optical signal from an endoscopic camera being used in conjunction with said port.

It is another object of the present invention to provide the method as defined above, additionally comprising step of inserting said port trans-orally and placing, while in use, partially within the upper hypopharynx and partially within the oral cavity, wherein said flexible port is further adapted to sit in place in a bent fashion.

It is another object of the present invention to provide the method as defined above, additionally comprising step of providing said flexible body with two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions.

It is another object of the present invention to provide the method as defined above, additionally comprising step of providing said intermediate portion in an accordion shape.

It is another object of the present invention to provide the method as defined above, additionally comprising step of providing said plurality of longitudinal channels with a central channel and four side channels.

It is another object of the present invention to provide the method as defined above, additionally comprising step of providing said central channel so as to accommodate at least one of an endoscopic camera and a light source.

It is another object of the present invention to provide the method as defined above, wherein at least one of said channels is adapted to accommodate at least one optical fiber so as to transmit light to the distal part of said flexible port.

It is another object of the present invention to provide the method as defined above, additionally comprising step of providing at least one said plurality of longitudinal channels with an O-ring groove about its inner circumference substantially near the proximal end.

It is another object of the present invention to provide the method as defined above, additionally comprising step of providing each of said plurality of longitudinal channels with a locking mechanism with gradable friction levels.

It is another object of the present invention to provide the method as defined above, additionally comprising step of providing at least one of said plurality of longitudinal channels so as to accommodate at least one surgical tool selected from a group consisting of scissors, forceps, injection needle, dissector, laser, suction, fiber optics, and any other required surgical instrument.

It is another object of the present invention to provide the method as defined above, additionally comprising step of providing said port with a locking mechanism adapted to fixate the orientation and angle of said flexible body.

It is another object of the present invention to provide the method as defined above, additionally comprising step of providing said locking mechanism is an integral part of said port.

It is another object of the present invention to provide the method as defined above, additionally comprising step of coupling said locking mechanism to said port by means selected from a group consisting of mechanical means glue, vacuum, clips, magnetic means, electrical means and any combination thereof.

It is another object of the present invention to provide the method as defined above, additionally comprising a step of translating at least one of said distal, proximal and body inflatable zones along the longitudinal axis of said port.

It is another object of the present invention to provide the method as defined above, wherein said at least one translatable inflatable zone rides in grooves in said body of said port.

It is another object of the present invention to provide the method as defined above, additionally comprising a step of disposing said grooves on said body of said port in at least one manner selected from a group consisting of longitudinally and spirally.

It is another object of the present invention to provide a flexible port, wherein said flexible port comprises:
a flexible body comprising a plurality of longitudinal channels extending therethrough;
a proximal inflatable zone adapted for inflation and deflation; said proximal inflatable zone disposed about the outer circumference of said body near the proximal end of said body, wherein the outer diameter of said proximal inflatable zone is no larger than that of said body when said proximal inflatable zone is in its deflated state, and further wherein said proximal inflatable zone is adapted to inflate to a predetermined outer circumference at least large enough to contact the inner walls of a body passage of a patient;
a distal inflatable zone adapted for inflation and deflation, said distal inflatable zone disposed about the outer circumference of said body near the distal end of said body, wherein the outer diameter of said distal inflatable zone is no larger than that of said body when said distal inflatable zone is in its deflated state, and further wherein said distal inflatable zone is adapted to inflate to a predetermined outer circumference at least large enough to contact the inner walls of said body passage of a patient.

It is another object of the present invention to provide the flexible port as defined above, wherein said body passage is the urethra.

It is another object of the present invention to provide the flexible port as defined above, wherein said body passage is the intestinal tract.

It is another object of the present invention to provide the flexible port as defined above, wherein said body passage is the vagina.

It is another object of the present invention to provide the flexible port as defined above to be used for endoscopic surgery of any other area of the body especially using the surgical approaches of NOTES (natural orifice tranluminal endoscopic surgery).

It is another object of the present invention to provide the flexible port as defined above, wherein said body passage is the ear.

It is another object of the present invention to provide the flexible port as defined above, wherein said distal inflatable zone comprises at least one balloon.

It is another object of the present invention to provide the flexible port as defined above, wherein said proximal inflatable zone comprises at least one balloon.

It is another object of the present invention to provide the flexible port as defined above, wherein said distal inflatable zone comprises at least two separate balloons, each of which is adapted to inflate so as to encircle part of said circumference.

It is another object of the present invention to provide the flexible port as defined above, wherein said proximal inflatable zone comprises at least two separate balloons, each of which is adapted to inflate so as to encircle part of said circumference.

It is another object of the present invention to provide the flexible port as defined above, wherein said proximal and distal inflatable zones are integral with said body of said port.

It is another object of the present invention to provide the flexible port as defined above, wherein substantially all of said body of said flexible port comprises at least one inflatable zone.

It is another object of the present invention to provide the flexible port as defined above, wherein substantially all of said flexible port comprises at least one inflatable zone.

It is another object of the present invention to provide the flexible port as defined above, additionally comprising a proximal inflation inlet located at the proximal end of said body, said proximal inflation inlet connectable to an external source of pressurized fluid.

It is another object of the present invention to provide the flexible port as defined above, additionally comprising a distal inflation inlet located at the proximal end of said body, said distal inflation inlet connectable to an external source of pressurized fluid.

It is another object of the present invention to provide the flexible port as defined above, wherein said external source of fluid is pressurized.

It is another object of the present invention to provide the flexible port as defined above, wherein said fluid is selected from a group consisting of air, nitrogen, argon, water, saline solution, anesthetic, analgesic, antibiotic, preservative, anti-caking agent, anti-sticking agent, and any combination thereof.

It is another object of the present invention to provide the flexible port as defined above, additionally comprising at least one first fluid connection channel providing a fluid connection between said proximal inflatable zone and said proximal inflation inlet

It is another object of the present invention to provide the flexible port as defined above, additionally comprising at least one second fluid connection channel providing a fluid connection between said distal inflatable zone and said distal inflation inlet.

It is another object of the present invention to provide the flexible port as defined above, wherein cross-sectional shape of said body comprises at least one selected from a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the flexible port as defined above, wherein n is less than about 20.

It is another object of the present invention to provide the flexible port as defined above, wherein said cross-sectional shape differs in different portions of said body.

It is another object of the present invention to provide the flexible port as defined above, wherein diameter of said body differs in different portions of said body.

It is another object of the present invention to provide the flexible port as defined above, wherein said diameter is greatest at said proximal end and smallest at said distal end of said body.

It is another object of the present invention to provide the flexible port as defined above, wherein said diameter is greater in at least one of said proximal end and said distal end of said body than in at least some portion of central region of said body.

It is another object of the present invention to provide the flexible port as defined above, wherein said fluid is selected from a group consisting of air, nitrogen, argon, water, saline solution, anesthetic, analgesic, antibiotic, preservative, anti-caking agent, anti-sticking agent, and any combination thereof.

It is another object of the present invention to provide the flexible port as defined above, wherein said longitudinal channels have cross-section selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the flexible port as defined above, wherein cross-section of said longitudinal channels differs in shape in different portions of said body.

It is another object of the present invention to provide the flexible port as defined above, wherein cross-section of said longitudinal channels differs in size in different portions of said body.

It is another object of the present invention to provide the flexible port as defined above, wherein said port is modular.

It is another object of the present invention to provide the flexible port as defined above, wherein said modules are selected from a group consisting of proximal portion modules, distal portion modules and body portion modules.

It is another object of the present invention to provide the flexible port as defined above, wherein said modules differ in size.

It is another object of the present invention to provide the flexible port as defined above, wherein said modules differ in shape.

It is another object of the present invention to provide the flexible port as defined above, adapted to be inserted intra-orally and to be placed, while in use, partially within the upper hypopharynx and partially within the oral cavity, wherein said flexible port is further adapted to sit in place in a bent fashion.

It is another object of the present invention to provide the flexible port as defined above, wherein said flexible body comprises two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions.

It is another object of the present invention to provide the flexible port as defined above, wherein said intermediate portion is formed in an accordion shape.

It is another object of the present invention to provide the flexible port as defined above, wherein said plurality of longitudinal channels comprises a central channel and four side channels.

It is another object of the present invention to provide the flexible port as defined above, wherein said central channel is adapted to accommodate at least one of an endoscopic camera and a light source.

It is another object of the present invention to provide the flexible port as defined above, wherein at least one of said channels is adapted to accommodate at least one optical fiber so as to transmit light to the distal part of said flexible port.

It is another object of the present invention to provide the flexible port as defined above, wherein at least one said plurality of longitudinal channels further comprises an O-ring groove about its inner circumference substantially near the proximal end.

It is another object of the present invention to provide the flexible port as defined above, wherein each of said plurality of longitudinal channels further comprises a locking mechanism with gradable friction levels.

It is another object of the present invention to provide the flexible port as defined above, wherein at least one of said plurality of longitudinal channels is adapted to accommodate at least one surgical tool selected from a group consisting of scissors, forceps, injection needle, dissector, laser, suction, fiber optics, and any other required surgical instrument.

It is another object of the present invention to provide the flexible port as defined above, additionally comprising a locking mechanism adapted to fixate the orientation and angle of said flexible body.

It is another object of the present invention to provide the flexible port as defined above, wherein said locking mechanism is an integral part of said port.

It is another object of the present invention to provide the flexible port as defined above, wherein said locking mechanism is coupled to said port by means selected from a group consisting of mechanical means glue, vacuum, clips, magnetic means, electrical means and any combination thereof.

It is another object of the present invention to provide the flexible port as defined above, additionally comprising a locking mechanism adapted to fixate at least one selected from a group consisting of (a) at least one of said channels; (b) said proximal inflatable zone; (c) said distal inflatable zone; (d) said entire port; and any combination thereof.

It is another object of the present invention to provide the flexible port as defined above, wherein at least one of said distal, proximal and body inflatable zones is enabled to be translated along the longitudinal axis of said port.

It is another object of the present invention to provide the flexible port as defined above, wherein said at least one translatable inflatable zone rides in grooves in said body of said port.

It is another object of the present invention to provide the flexible port as defined above, wherein said grooves are disposed on said body of said port in at least one manner selected from a group consisting of longitudinally and spirally.

It is another object of the present invention to provide a port system, comprising:
a flexible port as defined above; and,
a stand adapted to accommodate simultaneously the proximal ends of a plurality of surgical tools, said tools enabled to be passed through the channels of the flexible port.

It is another object of the present invention to provide a port system as defined above, wherein said stand is free-standing.

It is another object of the present invention to provide a port system as defined above, wherein said stand is adapted to be anchored to a surgical bed, and further comprising anchoring means for anchoring said stand to said surgical bed.

It is another object of the present invention to provide a port system as defined above, further comprising a screen adapted to display an optical signal from an endoscopic camera being used in conjunction with said port.

It is another object of the present invention to provide a method for using a flexible port, comprising;
obtaining a flexible port comprising:
   a flexible body comprising a plurality of longitudinal channels extending therethrough;
   a proximal inflatable zone adapted for inflation and deflation; said proximal inflatable zone disposed about the outer circumference of said body near the proximal end of said body, wherein the outer diameter of said proximal inflatable zone is no larger than that of said body when said proximal inflatable zone is in its deflated state, and further wherein said proximal inflatable zone is adapted to inflate to a predetermined outer circumference at least large enough to contact the inner walls of a body passage of a patient;
   a distal inflatable zone adapted for inflation and deflation, said distal inflatable zone disposed about the outer circumference of said body near the distal end of said body, wherein the outer diameter of said distal inflatable zone is no larger than that of said body when said distal inflatable zone is in its deflated state, and further wherein said distal inflatable zone is adapted to inflate to a predetermined outer circumference at least large enough to contact the inner walls of said body passage of a patient;
   a proximal inflation inlet located at the proximal end of said body, said proximal inflation inlet connectable to an external source of fluid;
   a distal inflation inlet located at the proximal end of said body, said distal inflation inlet connectable to an external source of fluid;
   a first fluid connection channel providing a fluid connection between said proximal inflatable zone and said proximal inflation inlet; and,
   a second fluid connection channel providing a fluid connection between said distal inflatable zone and said distal inflation inlet;
inserting said port into a body cavity or potential cavity of a patient;
connecting each of said inflation zones via said fluid connection channels and inflation inlets to at least one source of fluid;
inflating said inflation zones with said at least one fluid; and,
inserting at least one device selected from a group consisting of (a) an endoscopic camera, (b) a light source, and (c) at least one microsurgical instrument through at least one of said longitudinal channels.

It is another object of the present invention to provide the method as defined above, wherein said distal inflatable zone comprises at least one balloon.

It is another object of the present invention to provide the method as defined above, wherein said proximal inflatable zone comprises at least one balloon.

It is another object of the present invention to provide the method as defined above, wherein said distal inflatable zone comprises at least two separate balloons, each of which is adapted to inflate so as to encircle part of said circumference.

It is another object of the present invention to provide the method as defined above, wherein said proximal inflatable zone comprises at least two separate balloons, each of which is adapted to inflate so as to encircle part of said circumference.

It is another object of the present invention to provide the method as defined above, wherein said proximal and distal inflatable zones are integral with said body of said port.

It is another object of the present invention to provide the method as defined above, wherein substantially all of said body of said flexible port comprises at least one inflatable zone.

It is another object of the present invention to provide the method as defined above, wherein substantially all of said flexible port comprises at least one inflatable zone.

It is another object of the present invention to provide the method as defined above, additionally comprising a locking mechanism adapted to fixate at least one selected from a group consisting of (a) at least one of said channels; (b) said proximal inflatable zone; (c) said distal inflatable zone; (d) said entire port; and any combination thereof.

It is another object of the present invention to provide the method as defined above, wherein each of said plurality of longitudinal channels further comprises a locking mechanism with gradable friction levels, said method further comprising a step of locking at least one of said locking mechanisms, thereby fixing the position of the device inserted through said longitudinal channel.

It is another object of the present invention to provide the method as defined above, additionally comprising step of providing said cross-sectional shape of said body selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the method as defined above, wherein n is less than about 20.

It is another object of the present invention to provide the method as defined above, additionally comprising step of providing a different said cross-sectional shape in different portions of said body.

It is another object of the present invention to provide the method as defined above, additionally comprising step of providing a different diameter of said body in different portions of said body.

It is another object of the present invention to provide the method as defined above, additionally comprising step of providing said body with said diameter greatest at said proximal end of said body and smallest at said distal end of said body.

It is another object of the present invention to provide the method as defined above, additionally comprising step of providing said body with said diameter greater in at least one of said proximal end and said distal end of said body than in at least some portion of central region of said body.

It is another object of the present invention to provide the method as defined above, additionally comprising step of selecting said fluid from a group consisting of air, nitrogen, argon, water, saline solution, anesthetic, analgesic, antibiotic, preservative, anti-caking agent, anti-sticking agent, and any combination thereof.

It is another object of the present invention to provide the method as defined above, additionally comprising step of providing said inflation fluid as a pressurized gas.

It is another object of the present invention to provide the method as defined above, additionally comprising step of providing said longitudinal channels with cross-section selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

It is another object of the present invention to provide the method as defined above, additionally comprising step of providing cross-section of said longitudinal channels differing in shape in different portions of said body.

It is another object of the present invention to provide the method as defined above, additionally comprising step of providing cross-section of said longitudinal channels differing in size in different portions of said body.

It is another object of the present invention to provide the method as defined above, additionally comprising a step of assembling said flexible port from modules.

It is another object of the present invention to provide the method as defined above, wherein said modules are selected from a group consisting of proximal portion modules, distal portion modules and body portion modules.

It is another object of the present invention to provide the method as defined above, wherein said modules differ in size.

It is another object of the present invention to provide the method as defined above, wherein said modules differ in shape.

It is another object of the present invention to provide the method as defined above, further comprising a step of fixing said port to a stand adapted to accommodate simultaneously the proximal ends of a plurality of surgical tools, said tools enabled to be passed through the channels of the flexible port.

It is another object of the present invention to provide the method as defined above, further comprising a step of displaying on a screen an optical signal from an endoscopic camera being used in conjunction with said port.

It is another object of the present invention to provide the method as defined above, additionally comprising step of inserting said port trans-orally and placing, while in use, partially within the upper hypopharynx and partially within the oral cavity, wherein said flexible port is further adapted to sit in place in a bent fashion.

It is another object of the present invention to provide the method as defined above, additionally comprising step of providing said flexible body with two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions.

It is another object of the present invention to provide the method as defined above, additionally comprising step of providing said intermediate portion in an accordion shape.

It is another object of the present invention to provide the method as defined above, additionally comprising step of providing said plurality of longitudinal channels with a central channel and four side channels.

It is another object of the present invention to provide the method as defined above, additionally comprising step of providing said central channel so as to accommodate at least one of an endoscopic camera and a light source.

It is another object of the present invention to provide the method as defined above, wherein at least one of said channels is adapted to accommodate at least one optical fiber so as to transmit light to the distal part of said flexible port.

It is another object of the present invention to provide the method as defined above, additionally comprising step of providing at least one said plurality of longitudinal channels with an O-ring groove about its inner circumference substantially near the proximal end.

It is another object of the present invention to provide the method as defined above, additionally comprising step of providing each of said plurality of longitudinal channels with a locking mechanism with gradable friction levels.

It is another object of the present invention to provide the method as defined above, additionally comprising step of providing at least one of said plurality of longitudinal channels so as to accommodate at least one surgical tool selected from a group consisting of scissors, forceps, injection needle, dissector, laser, suction, fiber optics, and any other required surgical instrument.

It is another object of the present invention to provide the method as defined above, additionally comprising step of providing said port with a locking mechanism adapted to fixate the orientation and angle of said flexible body.

It is still an object of the present invention to provide the method as defined above, additionally comprising step of providing said locking mechanism is an integral part of said port.

It is lastly an object of the present invention to provide the method as defined above, additionally comprising step of coupling said locking mechanism to said port by means selected from a group consisting of mechanical means glue, vacuum, clips, magnetic means, electrical means and any combination thereof.

It is another object of the present invention to provide the method as defined above, additionally comprising a step of translating at least one of said distal, proximal and body inflatable zones along the longitudinal axis of said port.

It is another object of the present invention to provide the method as defined above, wherein said at least one translatable inflatable zone rides in grooves in said body of said port.

It is another object of the present invention to provide the method as defined above, additionally comprising a step of disposing said grooves on said body of said port in at least one manner selected from a group consisting of longitudinally and spirally.

### BRIEF DESCRIPTION OF THE DRAWINGS

The invention is now described with reference to the drawings, wherein

FIG. 1 presents views (not to scale) illustrating the construction of a flexible laryngoscope or flexible port according to two embodiments of the invention;

FIG. 2 presents a view (not to scale) of the proximal end of a flexible laryngoscope or flexible port according to one embodiment of the invention; and,

FIG. 3 presents a view (not to scale) of the distal end of the flexible laryngoscope or flexible port according to one embodiment of the invention.

FIGS. 4 & 5 provide another embodiment of the present invention in which a sliding mechanism is provided for the inflatable zones.

FIG. 6

[symbol]

FIG. 7 illustrates one example of the amount of articulation that can be obtained while using the flexible laryngoscope or flexible port of the present invention.

FIG. 8 illustrates a closer view of one of the balloon-like inflatable zones 120 which may glide along the port and the associated inflation tube.

FIG. 9 illustrates a stand adapted to accommodate simultaneously the proximal ends of a plurality of surgical tools, at times such that the tools are not in use. It is to be understood that the stand is an accessory to the laryngoscope or port and is not an integral part of the device.

### DETAILED DESCRIPTION OF THE PREFERRED EMBODIMENTS

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore the invention is not limited by that which is illustrated in the figures and described in the specification, but only as indicated in the accompanying claims, with the proper scope determined only by the broadest interpretation of said claims.

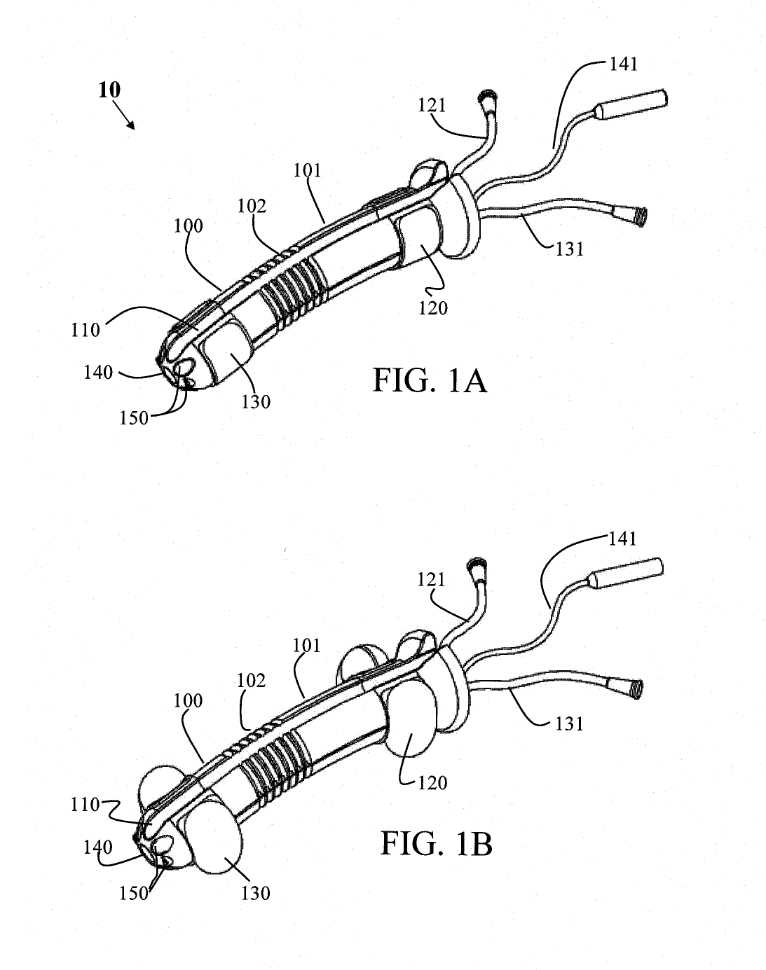

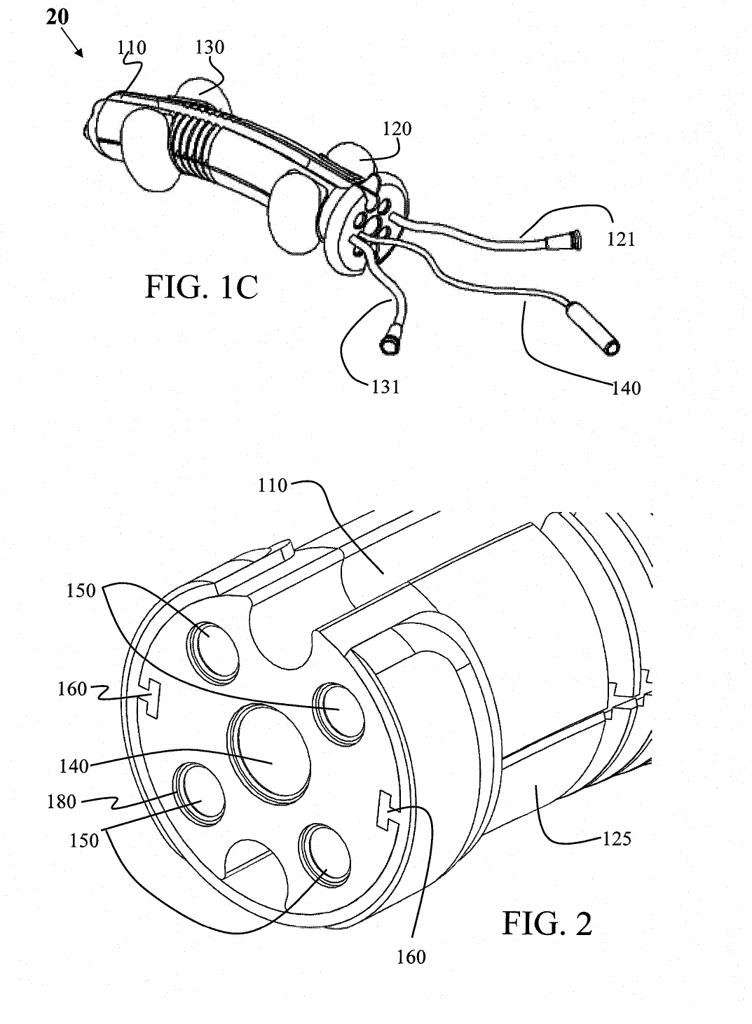

Reference is now made to FIG. 1, which illustrates (not to scale) the assembly of the flexible laryngoscope or flexible port of the present invention. FIG. 1A illustrates one preferred embodiment 10 of the invention. In this embodiment, the laryngoscope comprises a generally cylindrical body made of a biocompatible material such as plastic. The body is constructed to be flexible so that it can be bent while in place within the patient's oral cavity and hypopharynx. The diameter of the body is chosen to be appropriate for the size of the patient's oral cavity and throat, while the length is chosen to be appropriate for the patient and the type of medical procedure being performed. In preferred embodiments of the invention, the laryngoscope is provided in a variety of standard sizes.

In some embodiments, the laryngoscope or port comprises a body with cross-sectional shape selected from a group consisting of triangular, trapezoidal, rectangular, rhomboidal, polygonal, oval, and any combination thereof.

In some embodiments, the cross-sectional shape of the laryngoscope or port is similar throughout the length of the laryngoscope or port. In other embodiments, the cross-sectional shape differs in different parts of the laryngoscope or port. An illustrative example of an embodiment of a laryngoscope or port with similar cross-sections throughout is the embodiment 10 of the laryngoscope of FIG. 1A, which comprises a generally cylindrical cross-section throughout. In an illustrative example of another embodiment, the proximal end has generally circular cross-section, matching the shape of the oral cavity, the distal portion of the body has oval cross-section to minimize pressure on the larynx, the middle portion of the body has hexagonal cross-section to better control bending. These examples are illustrative; many other combinations of cross-section in different portions of the laryngoscope or port will be obvious to a person with ordinary skill in the art.

In the embodiment of FIG. 1A, the body has generally the same diameter throughout, if the diameter in a corrugated section is taken as the largest diameter in that corrugated section. In other embodiments, the diameter differs in different parts of the body, over and above the diameter variations required to form corrugations. In some embodiments, the distal end is wider than the proximal end so that the laryngoscope or port is generally conic, narrowing towards the distal end. In other embodiments, the laryngoscope or port is narrowest in some portion of the body, with the proximal and distal portions of the body wider than at least some of the central portion of the body. These examples are illustrative; other arrangements of will be obvious to a person with ordinary skill in the art.

In some embodiments, the material out of which the body of the laryngoscope or port is made is flexible in order to allow bending of the body while it is in place, in preferred embodiments, the body is constructed as illustrated in FIG. 1. In these embodiments, the body comprises three integrally formed sections: distal and proximal end portions (100 and 101, respectively) with smooth surfaces, and an intermediate portion 102 formed of flexible material. The distal and proximal end portions are substantially cylindrical along most of their lengths. In the preferred embodiment illustrated in FIG. 1, the proximal terminus of proximal end portion 101 is slightly flared, while the distal terminus 103 of distal end portion 100 has a general rounded shape. Each of the end portions further comprises a groove (not shown in FIG. 1) about its circumference adapted for insertion one of the balloon-like inflatable zones 120 and 130, as described in detail below. Intermediate portion 102 comprises a wall comprising a series of spaced corrugations having radial outer crests separated by intervening valleys, the greatest diameter of said radial outer crests being no greater than the largest diameter of said end portions.

In the most preferred embodiments, this intermediate portion is formed in a corrugated (accordion-fold) manner. Means for producing such corrugated flexible tubes are well-known in the art. The corrugations allow the intermediate portion to bend while maintaining the generally straight structures of the end portions.

Such an accordion-fold manner of the intermediate portion will provide the laryngoscope or port with mechanical properties enabling the same to flex and maneuver in much larger angles and orientations than a laryngoscope or port of the same materials and the same wall thickness without such corrugations.

However, it should be pointed out that the flexibility described above can also be obtained by altering the thickness and/or type of the material used for the manufacturing of the laryngoscope or port.

According to another embodiment of the present invention, such mechanical properties (namely, the articulation) are obtainable by altering the mechanical properties (namely, thickness and/or type) of at least one portion of said intermediate portion of the laryngoscope or port.

The body of the laryngoscope further comprises groove 110 along its length. The groove is adapted to allow an intubation tube to be placed within in order that the patient be able to continue breathing while the laryngoscope is in place.

According to another embodiment, there are two such grooves at 180 degrees to each other giving two options for positioning an endotracheal tube namely anterior or posterior glottis depending on desired surgical field.

According to another embodiment each inflation zone comprises 2 separate inflatable balloons; Two proximal and two distal.

Such division enables fine adjustment of the final position of the laryngoscope or port by selectively inflating the balloons until reaching the desired position/view/angle.

Such selective inflation provides a “steerable articulation” and expansion of the surgical field by lateralizing tissues and also anchoring and stabilizing the whole device.

According to another embodiment, at least one inflation zone is integral to the laryngoscope or port, so that no separate inflation balloons or other inflation means are needed. In some embodiments with integral inflation zones, the inflation zones exist in only portions of the laryngoscope or port, while in other embodiments, distention of the entire outer surface of the laryngoscope or port is possible. An embodiment of a laryngoscope or port with inflation zones enabling distention of the entire outer surface is a flexible balloon-like membrane forming a cover for the laryngoscope or port, said flexible outer membrane connected to the inner parts of the laryngoscope or port by sufficient connection means to prevent said inner parts of the laryngoscope or port from sliding or rotating against said flexible membrane. The outer membrane and the outer surface of the inner parts form at least one gas-tight volume, said at least one gas-tight volume is enabled to be filled in the same manner as the separate inflation balloons described hereinabove.

In another, preferred, embodiment, the entire laryngoscope or port is inflatable; the channels 150 and fiber optic channels taking light from connectors 140 forming inner walls for the at least one inflation zone, while the surface (100, 101, and possibly 102) and the at least one groove 110 form an outer wall. In some variations of this preferred embodiment, there is no corrugated zone, while in other variations, there is at least one corrugated zone (102). Said corrugated zone may be used to stabilize regions of curvature in inflatable ports.

In another embodiment, the body of the laryngoscope or port is inflatable, with at least one of the distal and proximal ends rigid or semi-rigid. In this embodiment, the channels 150 and fiber optic channels taking light from connectors 140 form inner walls for the at least one inflation zone, while the surface (100, 101, and possibly 102) and the at least one groove 110 form an outer wall. In some variations of this preferred embodiment, there is no corrugated zone, while in other variations, there is at least one corrugated zone (102). Said corrugated zone may be used to stabilize regions of curvature in laryngoscopes or ports with inflatable body.

In embodiments with a plurality of gas-tight volumes, said volumes may be disposed radially, longitudinally, axially, spirally, and any combination thereof.

In some embodiments, the at least one gas-tight volume is subdivided into chambers connected such that transfer of gas between chambers is significantly slower than the rate at which the at least one gas-tight chamber is filled. This relatively slow equalization of pressure between different chambers may be used to enable the surgeon to complete the operation in case of accidental rupture of any part of the outer membrane. It may also be used to “fine tune” the position of the laryngoscope or port.

In embodiments with the at least one inflation zone integral to the laryngoscope or port, the laryngoscope is inserted through the patient's mouth as described hereinbelow. After insertion, the laryngoscope is inflated as described hereinbelow. In some embodiments with an inflation zone comprising a plurality of gas-tight chambers, each chamber can be inflated separately, so that different pressures can be maintained in different portions of the inflation zone.

Embodiments with entirely inflatable body have the following advantages:
1. The diameter of the laryngoscope or port in the deflated state is much smaller than that of conventional laryngoscopes or ports, thereby allowing easier insertion and significantly reducing the potential for trauma to the delicate tissues of the larynx, throat and trachea.
2. After insertion, the body of the laryngoscope or port may be controllably inflated to a desired pressure, thereby retaining the laryngoscope or port stably in position while not exerting undue pressure on any portion of the larynx, throat or trachea.
3. Adequate exposure of the surgical field is maintained.
4. The laryngoscope or port is designed such that the channels (150) and the at least one groove (160) are built in to the inflatable body and are therefore available for use immediately after inflation of the body.
5. The laryngoscope or port is designed such that the fiber optics for illumination are built in to the inflatable body and are available for use immediately after inflation of the body. In some embodiments, the illumination channels provide sufficient stiffness to the inflatable body to allow easy insertion of the laryngoscope or port.

The body further comprises a plurality of longitudinal channels through the length of its interior. In preferred embodiments of the laryngoscope or port, it comprises a central channel and a plurality of channels 150 disposed circumferentially about the central channel. The diameter of the central channel is adapted for accommodation of an endoscopic camera and a light source. The diameters of the other channels are adapted to accommodate other microsurgical tools. In preferred embodiments, the channels further comprise locking mechanisms with gradable friction levels to allow the endoscopic camera and surgical tools to slide through the channels, and then to lock the camera and tools in place, enabling proper positioning of the tools and maintenance of the tools in their desired locations.

According to another embodiment, the central channel can be coupled to an external light source via coupler 140 and, for example, can enable the passage of optical fibers to the distal end of the same.

According to another embodiment of the present invention a locking mechanism is also provided to the flexible intermediate portion 102 so as to fix the orientation of the same.

According to one embodiment, the locking mechanism is a integral part of the laryngoscope or port and according to another embodiment the locking mechanism can be added and coupled to the laryngoscope or port.

According to one embodiment, the locking mechanism is coupled to the laryngoscope or port by mechanical means (e.g., glue, vacuum, clips etc.), magnetic means (e.g., magnetic filed), electrical means and any combination thereof.

According to another embodiment, the locking mechanism is adapted to perform at least one selected from a group consisting of:
fix at least one of the channels and to lock the same in place and in the position of endoscope or surgical tool.
secure the position of distal and/or proximal balloons which are situated on grooves to allow individual positioning by gliding movement.
lock the bend/articulation of laryngoscope or port.

The laryngoscope or port further comprises two inflatable zones, a proximal inflatable zone 120 located near the proximal end of the laryngoscope or port and a distal inflatable zone 130 located near the distal end of the laryngoscope or port. The balloon-like inflatable zones are made of a suitable biocompatible flexible material that expands on being filled with fluid. In a preferred embodiment, said fluid is air. In other embodiments, the fluid is an inert gas such as, but not limited to, nitrogen or argon. In yet other embodiments, it is oxygen. In yet other embodiments, a mixture of gases is used. In yet other embodiments, the gas mixture includes medically active materials, such as, but not limited to, anesthetics, analgesics or antibiotics. In further embodiments, the fluid is a liquid such as, but not limited to, water or saline solution. In some embodiments where the inflatable zone is filled with a liquid, the liquid includes medically active materials such as, but not limited to, anesthetics, analgesics or antibiotics. The fluid may also contain inert materials such as, but not limited to, stabilizers, anti-caking or anti-sticking agents, or preservatives.

In preferred embodiments of the invention, the inflatable zones are attached (e.g. by gluing) to a ring-like member, made of a relatively stiff biocompatible plastic, that is adapted to fit into the groove in which the inflatable zone sits. In most preferred embodiments, a pair of tabs are attached to the inner surface of the ring-like member, which are adapted to fit into slots 160 (not shown in FIG. 1), thus fixing the inflatable zone in place.

According to another embodiment of the present invention, the inflatable zones are an integral part of the laryngoscope or port.

FIG. 1A illustrates the laryngoscope with the inflatable zones in their uninflated state, and FIG. 1B illustrates the laryngoscope with the inflatable zones in their inflated state. In the uninflated state, the outer diameter of the inflatable zones is no greater than the largest diameter of the body, so that the laryngoscope can be inserted into the patient via the patient's oral cavity. In the inflated state, the outer diameter of the inflatable zones is sufficient to contact the inner surface of the patient's throat and/or hypopharynx. Each inflatable zone is in fluid connection with an inflation inlet (121 and 131, respectively) located at the proximal end of the laryngoscope. The inflation inlet is designed to allow introduction of air or other gas to inflate the inflatable zones and removal of air to deflate them. The inflation inlet is designed either to make a sealable connection with the gas source or to comprise a separate sealing means (e.g. by a valve). In the embodiment illustrated in FIG. 1, the inflation inlets terminate in a connection (e.g. a female LUER type connection) which is adapted for connection to a gas-tight syringe. Each inflatable zone is inflated with air injected from the syringe; as long as the LUER connection is maintained, the inflatable zones remain inflated. The inflatable zones can be deflated by detaching the syringe or by pulling back on the syringe plunger. Alternatively, any other means for inflating the inflatable zones known in the art may be used instead.

The exact positioning of the inflatable zones along the length of the body is not vital to the operation of the laryngoscope or port. Reference is now made to FIG. 1C, which illustrates a second embodiment 20 of the laryngoscope. In this embodiment, the distal inflatable zone, rather than being located near the distal cap (as shown in FIGS. 1A and 1B), is located substantially adjacent to the flexible intermediate portion 102.

According to another embodiment of the present invention, either one of the distal/proximal inflatable zone has the ability to slide along the longitudinal axis of the laryngoscope or port. Such an embodiment will allow exact positioning of the inflatable zones (namely, the balloons) by the surgeon.

Reference is now made to FIG. 2, which provides a close-up view of the proximal end of the laryngoscope according to one embodiment of the invention.

In this view, the proximal inflatable zone 120 has been removed to reveal the groove 125 about the circumference of the body in which the inflatable zone sits. Also shown in FIG. 2 are a pair of slots 160 machined to match the tabs that are attached to the interior surface of the ring-like member to which the inflatable zones are attached. In the embodiment shown in FIG. 2, each of the longitudinal channels 150 further comprises an O-ring groove 180. An O-ring of appropriate outer and inner diameters is fitted into the O-ring groove in order to provide friction to hold the surgical tool in place. In some embodiments, the inner diameter of the proximal end of the channel is machined to be somewhat larger than the inner diameter of the channel itself, and the O-ring groove cut into the proximal end such that when an O-ring is inserted, it abuts the portion of the channel at the point at which the inner diameter narrows, thus minimizing the likelihood that the O-ring will accidentally be pushed out of its groove and into the channel itself during insertion of the endoscopic camera or surgical tool.

It should be noted that according to one embodiment, the grooves as mentioned above are completely divided between each set of balloons allowing individual inflation and movement of all balloons.

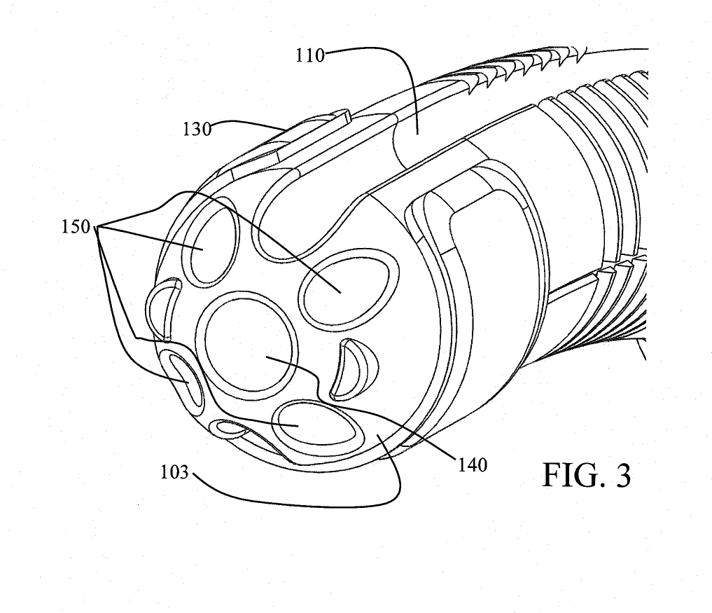

Reference is now made to FIG. 3, which provides a close-up view of the distal end of the laryngoscope according to one embodiment of the invention. In the embodiment shown in the drawing, the distal terminus 103 of the laryngoscope provides a generally rounded or capped shape to the distal end of the laryngoscope.

According to another embodiment of the present invention, the shape of distal end can have several possible options selected from triangular, rounded, oval, rectangular, regular shape, irregular shape, continuous shape, a polygon having n ribs, where n is greater than 1, and any combination thereof. Said ribs can be longitudinal, radial, spiral, and any combination thereof.

In the embodiment shown in FIG. 3, two of the channels 150 have a crescent moon-shaped cross-section. In this embodiment, these channels 150 have crescent moon shaped cross-sections throughout; in other embodiments, these channels 150 have crescent moon-shaped cross-section only at the distal end. In this embodiment, these crescent moon shaped channels are adapted to be light sources, receiving their light from an external light source attached to the proximal end of the laryngoscope.

The light source provided by these crescent-moon shaped channels may be a replacement for or in addition to a fiber optic light source running provided via one of the channels 150 with circular cross-section. These additional light channels need not have a crescent moon shaped cross section at the distal end. The cross section at the distal end may be circular, oval, lenticular, or polygonal with m sides, where m is a number greater than or equal to 3.

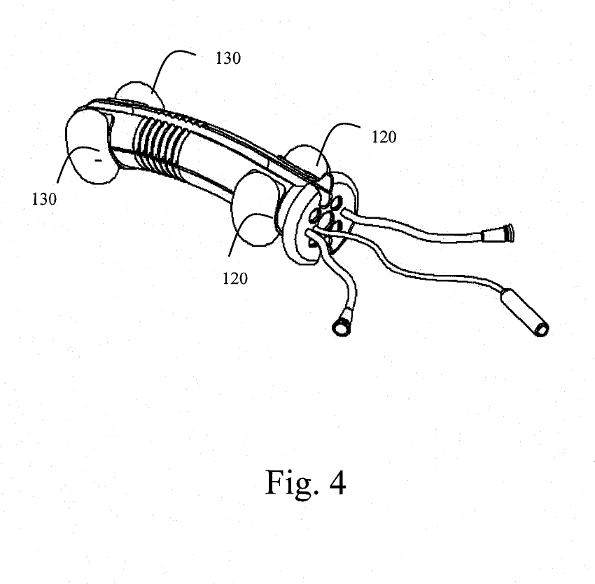

Reference is now made to FIGS. 4 and 5, which provides another embodiment of the present invention in which a sliding mechanism is provided such that either one of the balloon-like inflatable zones 120 and/or 130 can relocate their position along the laryngoscope or port main longitudinal axis.

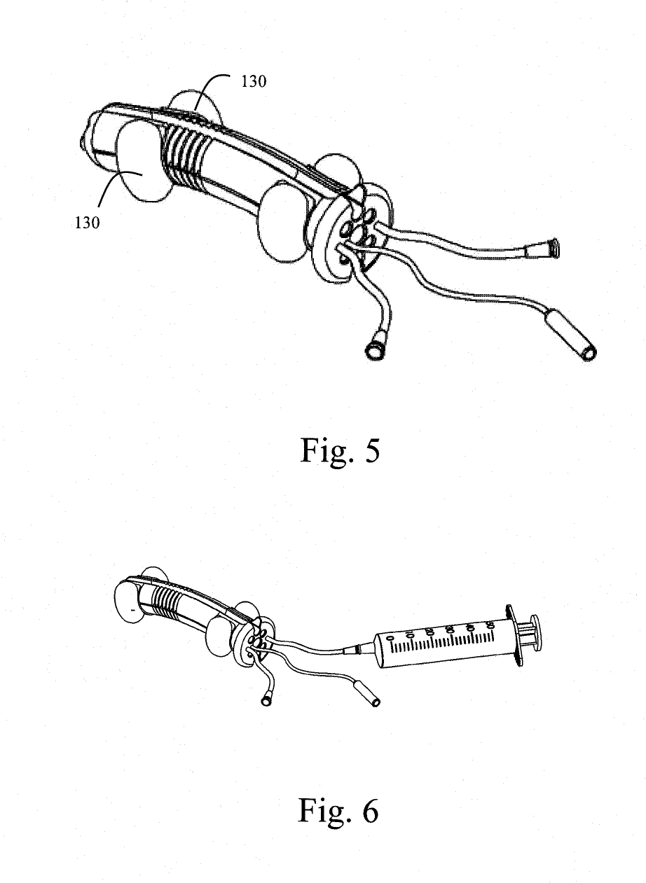

Reference is now made to FIG. 6 illustrating the [symbol] Inflation zones can be inflated individually, through separate inflation ports, or in groups containing a plurality of inflation zones. The inflation fluid is selected from a group consisting of air, nitrogen, argon, water, saline solution, anesthetic, analgesic, antibiotic, preservative, anti-caking agent, anti-sticking agent, and any combination thereof.

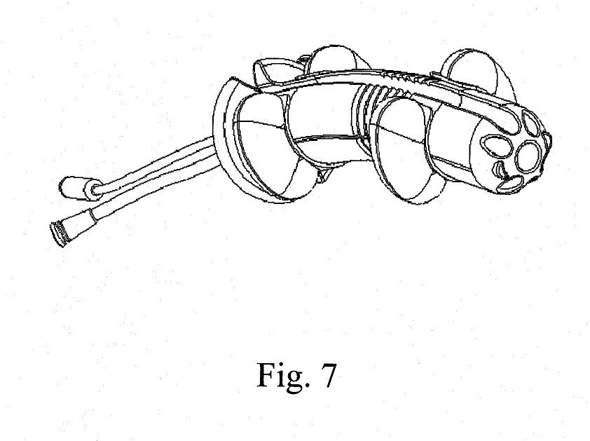

Reference is now made to FIG. 7 illustrating one example of the amount of articulation that can be obtained while using the flexible laryngoscope or port of the present invention.

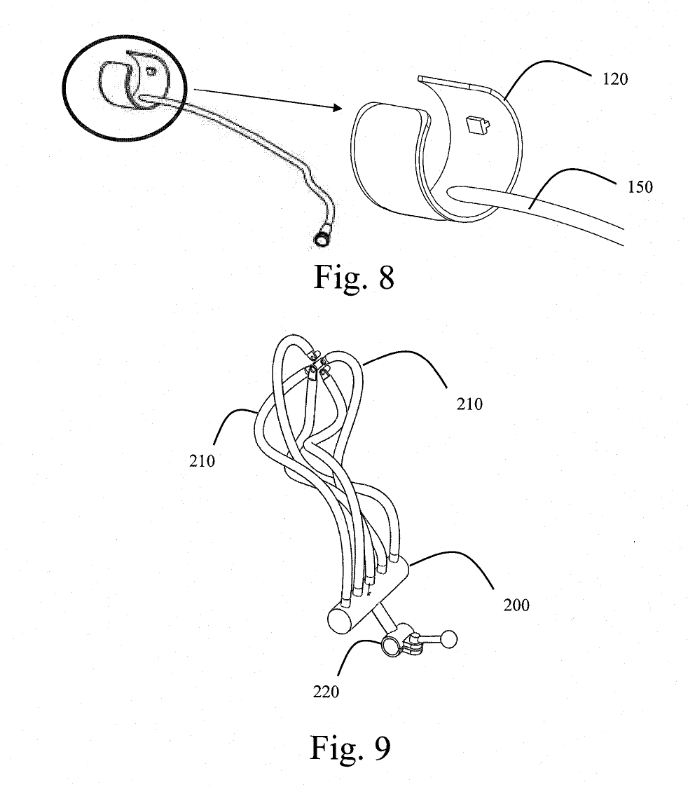

Reference is now made to FIG. 8 which illustrates a closer view of one of the inflation channels 150 and the balloon-like inflatable zones 120.

Reference is now made to FIG. 9 which illustrates an accessory to the laryngoscope, a stand (200) adapted to accommodate simultaneously the proximal ends of a plurality of surgical tools at such times as the tools are not in use. The accessory stand (200) is not an integral part of the laryngoscope or port, and the laryngoscope or port can be operated without use of the stand.

The stand comprises a number of flexible arms (210). Each of these flexible arms is meant to hold and support a tool or scope allowing the surgeon to manipulate more than two items by moving from one to another while not having to put down or hold ones not in present use.

In the embodiment shown in FIG. 9, the stand may be clamped to a rail by means of a clamping mechanism (220). Other means of supporting the stand will be obvious to one of ordinary skill in the art.

In another embodiment of the present invention, the laryngoscope or port has a modular design so that the size of the laryngoscope or port may be altered by adding or removing modules. Such modules can alter the length, width, or circumference of the laryngoscope or port as needed. Such a modular arrangement would mean that a smaller stock of laryngoscopes or ports is needed, as the operator assembles a laryngoscope or port of the size and shape needed from a stock of standard parts, rather than having many different sizes of laryngoscope or port.

In an embodiment of a laryngoscope or port with modular design, the laryngoscope or port may be lengthened or shortened by addition or removal of modules so that, for a non-limiting example, the same distal and proximal portions can be used both for surgery on the larynx and for surgery deep in the trachea. In such an embodiment, the operator could deflate a laryngoscope or port either partially or wholly, retract the laryngoscope or port sufficiently to remove a module, replace the laryngoscope or port and reinflate it, then continue with surgery at a position closer to the oral cavity which had previously been inaccessible.

In another embodiment of a laryngoscope or port with modular design, the cross-sectional shapes of the modules differ, with some being, for a non-limiting examples, of polygonal cross-section, others of lenticular cross section, and still others of circular cross-section.

In yet another embodiment, all cross-sectional shapes come in three sizes, “small”, “medium” and “large”, with modules of “small” cross-sections intended for use with children, modules of “medium” cross-section intended for use with women, and modules with “large” cross-section intended for use with men. Many other embodiments of sizing of the cross-sections will be obvious to one of ordinary skill in the art.

In yet another embodiment of a laryngoscope or port with modular design, modules fit outside other modules so that the “large” cross-section as described hereinabove would be built by surrounding a module of “small” cross-section with a ring-like module to create a “medium” cross-section, then surrounding said “medium” cross-section module with a larger ring-like module to create a “large” cross-section module. Many other embodiments of sizing of the cross-sections will be obvious to one of ordinary skill in the art.

Although the laryngoscope or port of the present design is drawn as slightly curved in the figures, this has been done for clarity. Embodiments of the laparoscope/port with rigid or semirigid sections between corrugated sections can have said rigid or semirigid sections either straight or curved, depending on the use of the laparoscope/port, with the degree of curvature adapted for the intended use. Fully-inflatable embodiments and embodiments with fully-inflatable body need have little intrinsic curvature in the deflated state, and may attain any curvature between straight (zero curvature) and a maximum which depends on the material of which the body is made. The curvature of fully-flexible embodiments and embodiments with fully flexible body, when inflated, can depend on the environment in which the laryngoscope or port is inflated. For example, if inflated within the body, the curvature can vary, approximately matching the curvature of the body portion surrounding a portion of the laryngoscope or port. For a non-limiting example, the curvature of said laryngoscope in the region of the larynx will differ from that of the same laryngoscope in the region of the oral cavity. The ability of the laryngoscope or port of the present invention to easily match its curvature to that of the body, without significant intervention by the operator, is one of the advantages of the laryngoscope or port of the present invention.

In some variants, embodiments of the fully-inflatable laryngoscopes or ports and embodiments with fully-inflatable body have portions such as, but not limited to, corrugated portions or separately-inflatable portions, with greater flexibility than other portions of said laryngoscopes or ports, enabling said laryngoscopes or ports to minimize discomfort to the patient in regions of higher curvature within the patient's body.

The device as described hereinabove has been adapted for use as a laryngoscope. In other embodiments, it can be adapted for use as a trochar or port system in other areas. Examples include, but are not limited to, laparoscopic surgery, intestinal surgery or examination where the point of entry is the anus, uterine surgery or examination where the point of entry is the vagina, bladder surgery or examination via the urethra and prostate surgery or examination via the urethra, and NOTES (Natural Orifice Transluminal Surgery).

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1-272. (canceled)

273. A flexible laryngoscope, wherein said flexible laryngoscope comprises:
a flexible body comprising a plurality of longitudinal channels extending therethrough and at least one longitudinal groove in outer circumference of said body, said longitudinal groove adapted to accommodate an intubation tube;
a proximal inflatable zone adapted for inflation and deflation; said proximal inflatable zone disposed about the outer circumference of said body near the proximal end of said body, wherein the outer diameter of said proximal inflatable zone is no larger than that of said body when said proximal inflatable zone is in its deflated state, and further wherein said proximal inflatable zone is adapted to inflate to a predetermined outer circumference at least large enough to contact the inner walls of the hypopharynx of a patient;
a distal inflatable zone adapted for inflation and deflation, said distal inflatable zone disposed about the outer circumference of said body near the distal end of said body, wherein the outer diameter of said distal inflatable zone is no larger than that of said body when said distal inflatable zone is in its deflated state, and further wherein said distal inflatable zone is adapted to inflate to a predetermined outer circumference at least large enough to contact the inner walls of the hypopharynx of a patient.

274. The flexible laryngoscope according to claim 273, wherein at least one of the following is being held true (a) said distal inflatable zone comprises at least one balloon; (b) said proximal inflatable zone comprises at least one balloon; (c) said distal inflatable zone comprises at least two separate balloons, each of which is adapted to inflate so as to encircle part of said circumference; (d) said proximal inflatable zone comprises at least two separate balloons, each of which is adapted to inflate so as to encircle part of said circumference; (e) said proximal and distal inflatable zones are integral with said body of said laryngoscope; (f) substantially all of said body of said flexible laryngoscope comprises at least one inflatable zone; (g) substantially all of said flexible laryngoscope comprises at least one inflatable zone; (h) said flexible laryngoscope additionally comprising a proximal inflation inlet located at the proximal end of said body, said proximal inflation inlet connectable to an external source of fluid; said fluid is selected from a group consisting of air, nitrogen, argon, water, saline solution, anesthetic, analgesic, antibiotic, preservative, anti-caking agent, anti-sticking agent, and any combination thereof; (i) said flexible laryngoscope additionally comprising a distal inflation inlet located at the proximal end of said body, said distal inflation inlet connectable to an external source of fluid; said fluid is selected from a group consisting of air, nitrogen, argon, water, saline solution, anesthetic, analgesic, antibiotic, preservative, anti-caking agent, anti-sticking agent, and any combination thereof.

275. The flexible laryngoscope according to claim 273, wherein at least one of the following is being held true (a) cross-sectional shape of said body comprises at least one selected from a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4; further wherein said cross-sectional shape either differs or substantially the same in different portions of said body; (b) the diameter of said body differs in different portions of said body; (c) said diameter is greatest at said proximal end and smallest at said distal end of said body; (d) said diameter is greater in at least one of said proximal end and said distal end of said body than in at least some portion of central region of said body; (e) said longitudinal channels have cross-section selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4; further wherein cross-section of said longitudinal channels is either substantially the same or differs in size or shape in different portions of said body; (f) said laryngoscope is modular; said modules are selected from a group consisting of proximal portion modules, distal portion modules and body portion modules; and any combination thereof.

276. The flexible laryngoscope according to claim 273, at least one of the following is being held true (a) said flexible laryngoscope is adapted to be inserted intra-orally and to be placed, while in use, partially within the upper hypopharynx and partially within the oral cavity, wherein said flexible laryngoscope is further adapted to sit in place in a bent fashion; (b) said flexible body comprises two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions; said intermediate portion is formed in an accordion shape; (c) said plurality of longitudinal channels comprises a central channel and four side channels; said central channel is adapted to accommodate an endoscopic camera and a light source; (d) at least one of said channels is adapted to accommodate at least one optical fiber so as to transmit light to the distal part of said flexible laryngoscope; and any combination thereof.

277. The flexible laryngoscope according to claim 273, wherein at least one of the following is being held true (a) each of said plurality of longitudinal channels further comprises a locking mechanism with gradable friction levels; (b) at least one of said plurality of longitudinal channels is adapted to accommodate at least one surgical tool selected from a group consisting of scissors, forceps, injection needle, dissector, laser, suction, and fiber optics or any other required surgical instrument; (c) said flexible laryngoscope additionally comprising a locking mechanism adapted to fixate the orientation and angle of said flexible body; wherein said locking mechanism is either coupled to or an integral part of said laryngoscope; (d) flexible laryngoscope additionally comprising a locking mechanism adapted to fixate at least one selected from a group consisting of (i) at least one of said channels; (ii) said proximal inflatable zone; (iii) said distal inflatable zone; (iv) said entire laryngoscope; and any combination thereof.

278. The flexible laryngoscope according to claim 273, additionally comprising a stand adapted to accommodate simultaneously the proximal ends of a plurality of surgical tools, said tools enabled to be passed through the channels of the flexible laryngoscope; wherein at least one of the following is being held true (a) said stand is free-standing; (b) said stand is adapted to be anchored to a surgical bed, and further comprising anchoring means for anchoring said stand to said surgical bed; (c) further comprising a screen adapted to display an optical signal from an endoscopic camera being used in conjunction with said laryngoscope; and any combination thereof.

279. A method for using a flexible laryngoscope, comprising:
obtaining a flexible laryngoscope comprising:
a flexible body comprising a plurality of longitudinal channels extending therethrough and at least one longitudinal groove in outer circumference of said body, said longitudinal groove adapted to accommodate an intubation tube;
a proximal inflatable zone adapted for inflation and deflation, said proximal inflatable zone disposed about the outer circumference of said body near the proximal end of said body, wherein the outer diameter of said proximal inflatable zone is no larger than that of said body when said proximal inflatable zone is in its deflated state, and further wherein said proximal inflatable zone is adapted to inflate to a predetermined outer circumference at least large enough to contact the inner walls of the hypopharynx of a patient;
a distal inflatable zone adapted for inflation and deflation, said distal inflatable zone disposed about the outer circumference of said body near the distal end of said body, wherein the outer diameter of said distal inflatable zone is no larger than that of said body when said distal inflatable zone is in its deflated state, and further wherein said distal inflatable zone is adapted to inflate to a predetermined outer circumference at least large enough to contact the inner walls of the hypopharynx of a patient;
a proximal inflation inlet located at the proximal end of said body, said proximal inflation inlet connectable to an external source of fluid;
a distal inflation inlet located at the proximal end of said body, said distal inflation inlet connectable to an external source of fluid;
a first fluid connection channel providing a fluid connection between said proximal inflatable zone and said proximal inflation inlet; and,
a second fluid connection channel providing a fluid connection between said distal inflatable zone and said distal inflation inlet;
inserting said laryngoscope trans-orally into the oral cavity and hypopharynx of a patient;
intubating said patient by inserting a endotracheal breathing tube and then gliding the laryngoscope into place with the endotracheal tube located in said longitudinal groove;
connecting each of said inflation zones via said fluid connection channels and inflation inlets to at least one source of fluid;
inflating said inflation zones with said at least one fluid; and,
inserting at least one device selected from a group consisting of (a) an endoscopic camera, (b) a light source, and (c) at least one microsurgical instrument through at least one of said longitudinal channels.

280. The method according to claim 279, wherein at least one of the following is being held true (a) said distal inflatable zone comprises at least one balloon; (b) said proximal inflatable zone comprises at least one balloon; (c) said distal inflatable zone comprises at least two separate balloons, each of which is adapted to inflate so as to encircle part of said circumference; (d) said proximal inflatable zone comprises at least two separate balloons, each of which is adapted to inflate so as to encircle part of said circumference; (e) said proximal and distal inflatable zones are integral with said body of said laryngoscope; (f) said flexible laryngoscope additionally comprising a locking mechanism adapted to fixate at least one selected from a group consisting of (i) at least one of said channels; (ii) said proximal inflatable zone; (iii) said distal inflatable zone; (iv) said entire laryngoscope; and any combination thereof; (g) each of said plurality of longitudinal channels further comprises a locking mechanism with gradable friction levels, said method further comprising a step of locking at least one of said locking mechanisms, thereby fixing the position of the device inserted through said longitudinal channel; and any combination thereof.

281. The method according to claim 279, additionally comprising at least one step selected from a group consisting of (a) providing said cross-sectional shape of said body selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4; (b) additionally comprising step of providing a different said cross-sectional shape in different portions of said body; (c) additionally comprising step of providing a different diameter of said body in different portions of said body; (c) providing said body with said diameter greatest at said proximal end of said body and smallest at said distal end of said body; (d) providing said body with said diameter greater in at least one of said proximal end and said distal end of said body than in at least some portion of central region of said body; (e) providing said longitudinal channels with cross-section selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4; (f) fixing said laryngoscope to a stand adapted to accommodate simultaneously the proximal ends of a plurality of surgical tools, said tools enabled to be passed through the channels of the flexible laryngoscope; (g) displaying on a screen an optical signal from an endoscopic camera being used in conjunction with said laryngoscope; (h) inserting said laryngoscope trans-orally and placing, while in use, partially within the upper hypopharynx and partially within the oral cavity, wherein said flexible laryngoscope is further adapted to sit in place in a bent fashion; and any combination thereof.

282. The method according to claim 279, additionally comprising at least one step selected from a group consisting of (a) providing said flexible body with two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions; (b) providing said intermediate portion in an accordion shape; (c) providing said plurality of longitudinal channels with a central channel and four side channels; (d) providing said central channel so as to accommodate at least one of an endoscopic camera and a light source.

283. The method according to claim 279, additionally comprising at least one step selected from a group consisting of (a) providing each of said plurality of longitudinal channels with a locking mechanism with gradable friction levels; (b) providing at least one of said plurality of longitudinal channels so as to accommodate at least one surgical tool selected from a group consisting of scissors, forceps, injection needle, dissector, laser, suction, and fiber optics or any other required surgical instrument; (c) providing said laryngoscope with a locking mechanism adapted to fixate the orientation and angle of said flexible body; (d) providing said locking mechanism is an integral part of said laryngoscope; (e) coupling said locking mechanism to said laryngoscope by means selected from a group consisting of mechanical means glue, vacuum, clips, magnetic means, electrical means and any combination thereof.

284. A flexible port for use during a laparoscopic surgery, wherein said flexible port comprises:
a flexible body comprising a plurality of longitudinal channels extending therethrough;
at least two inflatable zones adapted for inflation and deflation; one said inflatable zone distal to the other said inflatable zone, said inflatable zones disposed about the outer circumference of said body wherein the outer diameter of said distal inflatable zone is no larger than that of said body when said distal inflatable zone is in its deflated state, and further wherein said proximal and distal inflatable zones are adapted to inflate to a predetermined outer circumference at least large enough to retain said flexible port in a substantially fixed position relative to a patient's body.

285. The flexible port according to claim 284, wherein at least one of the following is being held true (a) said distal inflatable zone comprises at least one balloon; said proximal inflatable zone comprises at least one balloon; (c) said distal inflatable zone comprises at least two separate balloons, each of which is adapted to inflate so as to encircle part of said circumference; (d) said proximal inflatable zone comprises at least two separate balloons, each of which is adapted to inflate so as to encircle part of said circumference; (e) said proximal and distal inflatable zones are integral with said body of said port; (f) substantially all of said body of said flexible port comprises at least one inflatable zone; (h) said flexible port additionally comprising a proximal inflation inlet located at the proximal end of said body, said proximal inflation inlet connectable to an external source of pressurized fluid; said fluid is selected from a group consisting of air, nitrogen, argon, water, saline solution, anesthetic, analgesic, antibiotic, preservative, anti-caking agent, anti-sticking agent, and any combination thereof; (i) said flexible port additionally comprising a distal inflation inlet located at the proximal end of said body, said distal inflation inlet connectable to an external source of fluid; said fluid is selected from a group consisting of air, nitrogen, argon, water, saline solution, anesthetic, analgesic, antibiotic, preservative, anti-caking agent, anti-sticking agent, and any combination thereof.

286. The flexible port according to claim 284, wherein at least one of the following is being held true (a) cross-sectional shape of said body comprises at least one selected from a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4; further wherein said cross-sectional shape either substantially the same or differs in different portions of said body; (b) said diameter is greatest at said proximal end and smallest at said distal end of said body; (c) said diameter is greater in at least one of said proximal end and said distal end of said body than in at least some portion of central region of said body; (d) said longitudinal channels have cross-section selected from at least one of a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4; further wherein cross-section of said longitudinal channels is either substantially the same or differs in shape in different portions of said body; (e) said port is modular; said modules are selected from a group consisting of proximal portion modules, distal portion modules and body portion modules; said modules are substantially the same or differ in size or shape; (f) said flexible port is adapted to be inserted intra-orally and to be placed, while in use, partially within the upper hypopharynx and partially within the oral cavity, wherein said flexible port is further adapted to sit in place in a bent fashion; (g) said flexible body comprises two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions; (h) said plurality of longitudinal channels comprises a central channel and four side channels; said central channel is adapted to accommodate at least one of an endoscopic camera and a light source; (i) at least one of said channels is adapted to accommodate at least one optical fiber so as to transmit light to the distal part of said flexible port; and any combination thereof.

287. The flexible port according to claim 284, wherein at least one of the following is being held true (a) each of said plurality of longitudinal channels further comprises a locking mechanism with gradable friction levels; (b) at least one of said plurality of longitudinal channels is adapted to accommodate at least one surgical tool selected from a group consisting of scissors, forceps, injection needle, dissector, laser, suction, fiber optics, and any other required surgical instrument; (c) additionally comprising a locking mechanism adapted to fixate the orientation and angle of said flexible body; further wherein said locking mechanism is either coupled to or an integral part of said port; (d) said flexible port additionally comprising a locking mechanism adapted to fixate at least one selected from a group consisting of (i) at least one of said channels; (ii) said proximal inflatable zone; (iii) said distal inflatable zone; (iv) said entire port; and any combination thereof.

288. The flexible port according to claim 284, additionally comprising:
a stand adapted to accommodate simultaneously the proximal ends of a plurality of surgical tools, said tools enabled to be passed through the channels of the flexible port; at least one of the following is being held true (a) said stand is free-standing; (b) said stand is adapted to be anchored to a surgical bed, and further comprising anchoring means for anchoring said stand to said surgical bed; (c) further comprising a screen adapted to display an optical signal from an endoscopic camera being used in conjunction with said port.

289. A method for using a flexible port, comprising:
a flexible body comprising a plurality of longitudinal channels extending therethrough;
at least two inflatable zones adapted for inflation and deflation; one said inflatable zone distal to the other said inflatable zone, said inflatable zones disposed about the outer circumference of said body wherein the outer diameter of said distal inflatable zone is no larger than that of said body when said distal inflatable zone is in its deflated state, and further wherein said proximal and distal inflatable zones are adapted to inflate to a predetermined outer circumference at least large enough to retain said flexible port in a substantially fixed position relative to a patient's body;
a proximal inflation inlet located at the proximal end of said body, said proximal inflation inlet connectable to an external source of fluid;
a distal inflation inlet located at the proximal end of said body, said distal inflation inlet connectable to an external source of fluid;
a first fluid connection channel providing a fluid connection between said proximal inflatable zone and said proximal inflation inlet; and,
a second fluid connection channel providing a fluid connection between said distal inflatable zone and said distal inflation inlet;
inserting said port into a body cavity or potential cavity of a patient;
connecting each of said inflation zones via said fluid connection channels and inflation inlets to at least one source of fluid;
inflating said inflation zones with said at least one fluid; and,
inserting at least one device selected from a group consisting of (a) an endoscopic camera, (b) a light source, and (c) at least one microsurgical instrument through at least one of said longitudinal channels.

290. The method according to claim 289, wherein at least one of the following is being held true (a) said distal inflatable zone comprises at least one balloon; (b) said proximal inflatable zone comprises at least one balloon; (c) said distal inflatable zone comprises at least two separate balloons, each of which is adapted to inflate so as to encircle part of said circumference; (d) wherein said proximal inflatable zone comprises at least two separate balloons, each of which is adapted to inflate so as to encircle part of said circumference; (e) said proximal and distal inflatable zones are integral with said body of said port; (f) substantially all of said body of said flexible port comprises at least one inflatable zone.

291. The method according to claim 289, wherein at least one of the following is being held true (a) said flexible port additionally comprising a locking mechanism adapted to fixate at least one selected from a group consisting of (i) at least one of said channels; (ii) said proximal inflatable zone; (iii) said distal inflatable zone; (iv) said entire port; and any combination thereof; (b) each of said plurality of longitudinal channels further comprises a locking mechanism with gradable friction levels, said method further comprising a step of locking at least one of said locking mechanisms, thereby fixing the position of the device inserted through said longitudinal channel; (c) the cross-section of said longitudinal channels is selected from a group consisting of substantially circular, substantially lenticular, substantially oval, substantially crescent moon-shaped, substantially half moon-shaped, substantially triangular, substantially rectangular, substantially trapezoidal, substantially diamond-shaped, substantially rhomboidal, and substantially polygonal with n sides, where n is greater than 4.

292. The method according to claim 289, further comprising at least one step selected from a group consisting of (a) fixing said port to a stand adapted to accommodate simultaneously the proximal ends of a plurality of surgical tools, said tools enabled to be passed through the channels of the flexible port; (b) displaying on a screen an optical signal from an endoscopic camera being used in conjunction with said port; (c) inserting said port trans-orally and placing, while in use, partially within the upper hypopharynx and partially within the oral cavity, wherein said flexible port is further adapted to sit in place in a bent fashion; (d) providing said flexible body with two end portions having smooth surfaces and an intermediate portion made of flexible material, said intermediate portion formed integrally with said end portions, the wall of said intermediate portion comprising a series of spaced corrugations having radial outer crests separated by intervening valleys, the greatest diameter of said radial outer crests being no greater than the largest diameter of said end portions; (e) providing said plurality of longitudinal channels with a central channel and four side channels; (f) providing said central channel so as to accommodate at least one of an endoscopic camera and a light source; (g) providing each of said plurality of longitudinal channels with a locking mechanism with gradable friction levels; (h) providing at least one of said plurality of longitudinal channels so as to accommodate at least one surgical tool selected from a group consisting of scissors, forceps, injection needle, dissector, laser, suction, fiber optics, and any other required surgical instrument; (i) providing said port with a locking mechanism adapted to fixate the orientation and angle of said flexible body; (j) providing said locking mechanism either coupled to or as an integral part of said port; and any combination thereof.
